(19) United States
(12) Patent Application Publication
(10) Pub. No.: US 2024/0161913 A1
(43) Pub. Date: May 16, 2024

(54) PRECISION BIOLOGY SEARCH WITH MACHINE LEARNING AND DIGITAL TWIN TECHNOLOGY

(71) Applicant: TWIN HEALTH, INC., Mountain View, CA (US)

(72) Inventors: Jahangir MOHAMMED, Los Gatos, CA (US); Terrence Chun Yin POON, Foster City, CA (US); Mohamed THAJUDEEN, Tirunelveli (IN); Mulk Abdul Maluk MOHAMED, Tirunelveli (IN); Lisa Madhukanta Shah, Bethesda, MD (US)

(21) Appl. No.: 18/508,197

(22) Filed: Nov. 13, 2023

(30) Foreign Application Priority Data
Nov. 14, 2022 (IN) 202241065227

(51) Int. Cl. G16H 40/20; G06F 40/205

(52) U.S. Cl. CPC G16H 40/20; G06F 40/205

(57) ABSTRACT

A health search system receives a search query requesting a search for biological information specific to a patient. The health search system accesses a health profile of a patient storing biomarkers determined for the patient. The health search system searches one or more hierarchical data structures to identify one or more health parameters related to the search query where each node of the data structures corresponds to a health parameter belonging to the biological ontology. The health search system determines measurements for one or more biomarkers stored within the health profile of the patient corresponding to the identified health parameters. The health search system generates search results for the search query comprising the identified health parameters and the determined measurements corresponding to each health parameter. The health search system transmits the search results to the user device for display via a graphical user interface.

### CROSS-REFERENCE TO RELATED APPLICATIONS

This application claims the benefit of IN Provisional Application No. 202241065227, filed on Nov. 14, 2022, which is incorporated by reference in its entirety.

### BACKGROUND

### Field of Art

The disclosure relates generally to a patient health management platform, and more specifically, to a personalized health search platform for monitoring the metabolic health of a patient.

Human beings are arguably the most complex organisms on this planet. The human body is a single structure made up of cells, tissue, organs and organ systems. Human cells and tissues adapt to internal metabolic demands in many ways, mostly in response to hormones and/or nervous stimuli. The health of the human body may be affected by a combination of various biosignals such as oxygen, sleep, physical activities, and nutrients from food. Managing a patient's metabolic health is complicated because of how different factors such as nutrition, hormones, and external stimuli (e.g., pathogens and stress) continuously affect the patient's metabolism. However, conventional health management platforms do not monitor the human body continuously or measure the extent of the metabolic damage caused by a particular biosignal or combination of biosignals.

In addition, conventional platforms offer no means for a patient to access holistic information related to their metabolic health. Some conventional systems allow patients to search medical conditions or biological parameters to gain generic knowledge, but no such search engine allows a patient to gain personalized insights into their own complex metabolic condition. Additionally, patients are unable to submit precise queries to a platform regarding a a patient's unique metabolic conditions or behaviors.

### SUMMARY

A precision health search system performs a biological information search using a digital representation or digital twin of a patient. The system intelligently conducts a search related to the patient, including searching for biological parameters and medical conditions, based on a digital twin model. The precision health searching may be a system, a method, or a computer-readable storage medium that stores instructions that when executed by a processor cause the processor to perform certain steps.

In one embodiment, a precision health search system receives a search query requesting a search for biological information specific to a patient. The precision health search system accesses a health profile of a patient storing biomarker measurements determined for the patient based on biosignals collected for the patient over a given time period. The precision health search system searches one or more hierarchical data structures to identify one or more health parameters related to the search query. Each hierarchical data structure comprises a plurality of nodes organized into one or more levels of a biological ontology related to the search query. Each node of the ontology corresponds to a health parameter belonging to the biological ontology. The precision health search system determines measurements for one or more biomarkers stored within the health profile of the patient corresponding to the one or more identified health parameters. The precision health search system generates a set of search results for the search query comprising the identified one or more health parameters related to the search query and the determined biomarkers corresponding to each health parameter. The precision health search system transmits the search results to the user device as a response to the search query.

In another embodiment, a precision health search system receives a search query requesting a search for biological information specific to a patient. The precision health search system accesses a health profile of a patient storing biomarker measurements determined for the patient based on biosignals collected for the patient over a given time period. The precision health search system searches one or more hierarchical data structures to identify one or more medical conditions related to the search query. Each hierarchical data structure comprises a plurality of nodes organized into one or more levels of a biological ontology related to the search query. Each node of the ontology corresponds to a medical condition belonging to the biological ontology. The precision health search system determines measurements for one or more biomarkers stored within the health profile of the patient corresponding to the one or more identified health parameters. The precision health search system determines a patient-specific risk score by inputting the determined measurements to machine-learning models trained to predict patient-specific risk scores. The precision health search system generates a set of search results for the search query comprising the identified one or more medical conditions related to the search query and the patient-specific risk scores. The precision health search system transmits the search results to the user device as a response to the search query.

The disclosed systems and methods provide several technical advantages and improvement upon existing technology. As mentioned above, a human body is a complex system that requires a balanced combination of input parameters. When the balanced combination of input parameters is not properly offered to the body, the body starts to gradually impact the functionality of the system. The dynamic and complex nature of the correlation between the input to the human body and its behaviors makes it challenging to find a solution for health deterioration. However, the disclosed precision health search system enables the implementation of a monitoring system for the human body that is vital for the early diagnosis and treatment of diseases. The disclosed precision health search system may further provide user interface including visualization of correlation between the input parameters and the performance of systems of the human body.

Furthermore, the precision health search system provides a continuously updated search engine. Traditionally, the human body is monitored by methods such as radiological investigations, in vitro diagnostics and in vivo diagnostics. However, because human conditions are dynamically changing, conventional systems lack precise continuous knowledge of the human body. The above-mentioned methods provide only an instance of the condition, while the condition continues to dynamically change. The disclosed precision health search system may allow clinicians to continuously monitor the systems in the human body system, which is vital for the early detection of the root cause of the problem and treatment of diseases.

### BRIEF DESCRIPTION OF DRAWINGS

FIG. 1 shows a metabolic health manager 100 for monitoring a patient's metabolic health, for performing analytics on metabolic health data recorded for the patient, and for generating a patient-specific recommendation for treating any metabolic health-related concerns, according to one embodiment.

FIG. 2 is a block diagram of the system architecture of the patient health management platform 130 that includes a precision health search module 260, according to one embodiment.

FIG. 3 illustrates a block diagram of a precision health search module, according to one embodiment.

FIG. 4 illustrates a block diagram of a biology parameter search module 320, according to one embodiment.

FIG. 5A illustrates an example set of branches within the biosignal ontology 411, according to one embodiment.

FIG. 5B illustrates an example set of branches within the nutrition ontology 412, according to one embodiment.

FIG. 6 illustrates an example user interface displaying the results of a biology parameter search, according to one embodiment.

FIG. 7 illustrates a block diagram of a medical condition search module 330, according to one embodiment.

FIG. 8 illustrates an example set of branches within the medical condition ontology including multiple sublevels and nodes, according to one embodiment.

FIG. 9 illustrates an example process for performing a biological parameter search, according to one embodiment.

FIG. 10 illustrates an example process for performing a medical condition search, according to one embodiment.

FIG. 11 is a high-level block illustrating an example of a computing device used in either as a client device, application server, and/or database server, according to one embodiment.

The figures depict various embodiments of the presented invention for purposes of illustration only. One skilled in the art will readily recognize from the following discussion that alternative embodiments of the structures and methods illustrated herein may be employed without departing from the principles described herein.

### DETAILED DESCRIPTION

### System Environment

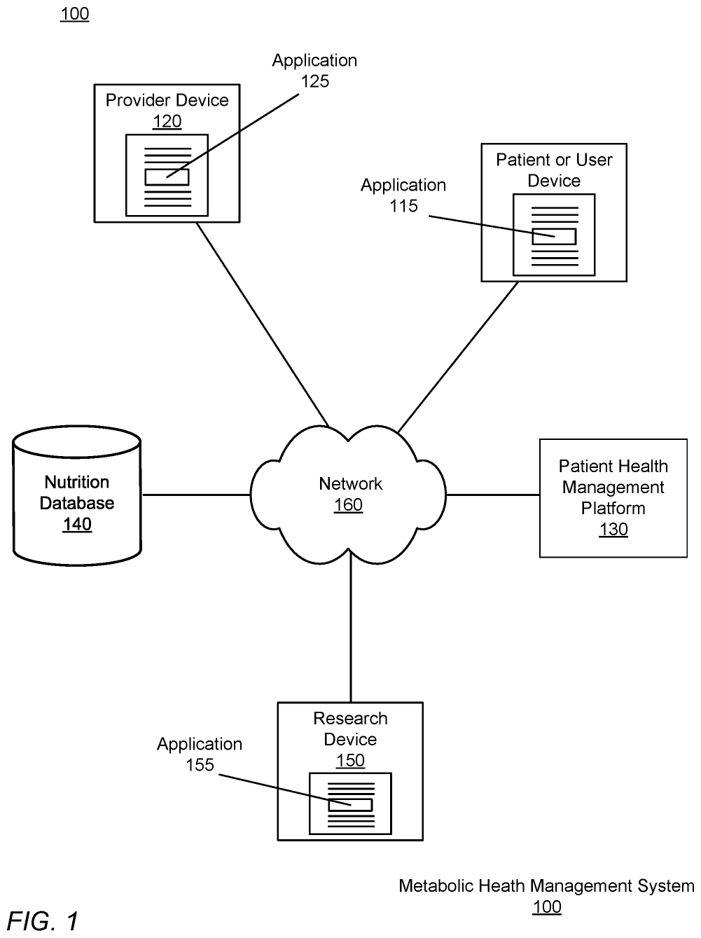

FIG. 1 shows a metabolic health manager 100 for monitoring a patient's metabolic health, for performing analytics on metabolic health data recorded for the patient, and for generating a patient-specific recommendation for treating any metabolic health-related concerns, according to one embodiment. The metabolic health manager 100 includes patient device(s) 110 (also referred to as user devices 110), provider device(s) 120, a patient health management platform 130, a nutrition database 140, research device(s) 150 and a network 160. However, in other embodiments, the system 100 may include different and/or additional components. For example, the patient device 110 can represent thousands or millions of devices for patients (e.g., patient mobile devices) that interact with the system in locations around the world. Similarly, the provider device 120 can represent thousands or millions of devices of providers (e.g., mobile phones, laptop computers, in-provider-office recording devices, etc.). In some cases, a single provider may have more than one device that interacts with the platform 130.

The patient device 110 is a computing device with data processing and data communication capabilities that is capable of receiving inputs from a patient. As explained above, in some embodiments, a user uses the health management platform with a patient device 110 to determine information about a patient. In other embodiments, the user and patient are the same person, in which case the user uses a patient device to determine information about their own health. Thus, it is understood that the terms “user” and “patient” may be used herein interchangeably to refer to the same person or to different people. In addition to data processing, the patient device 110 may include functionality that allows the device 110 to record speech responses articulated by a patient operating the device (e.g., a microphone), and to graphically present data to a patient (e.g., a graphics display). Examples of the patient device 110 include desktop computers, laptop computers, portable computers, GOOGLE HOME, AMAZON ECHO, etc. The patient device 110 may present information generated by the communication platform 130 via a mobile application configured to display and record patient responses. For example, a patient may receive a recommendation or an update regarding their metabolic health through an application 115.

The application 115 provides a user interface, herein referred to as a “patient dashboard,” that is displayed on a screen of the patient device 110. The patient dashboard allows a patient to input commands to control operation of the application 115. The patient dashboard enables users to track and manage changes in a patient's metabolic health. For example, the dashboard allows patients or other users to conduct searches, observe changes in a patient's metabolic health over time, receive recommendation notifications, exchange messages about treatment with a health care provider, etc. The application 115 may be coded as a web page, a series of web pages, or content otherwise coded to render within an internet browser. The application 115 may also be coded as a proprietary application configured to operate on the native operating system of the patient device 110. In addition to providing the dashboard, application 115 may also perform some data processing on biological and food data locally using the resources of patient device 110 before sending the processed data through the network 160. Patient data sent through the network 160 is received by the patient health management platform 130 where it is analyzed and processed to generate patient-specific health insights.

Similarly, a provider device 120 is a computing device with data processing and data communication capabilities that is capable of receiving inputs from a provider. The provider device 120 is configured to present a patient's medical history or medically relevant data (i.e., a display screen). The above description of the functionality of the patient device 110 also can apply to the provider device 120. The provider device 120 can be a personal device (e.g., phone, tablet) of the provider, a medical institution computer (e.g., a desktop computer of a hospital or medical facility), etc. In addition, the provider device 120 can include a device that sits within the provider office such that the patient can interact with the device inside the office. In such implementations, the provider device may be a customized device with audio and/or video capabilities (e.g., a microphone for recording, a display screen for text and/or video, an interactive user interface, a network interface, etc.). The provider device 120 may also present information to medical providers or healthcare organizations via a mobile application similar to the application described with reference to patient device 110.

Application 125 provides a user interface, herein referred to as a “provider dashboard,” that is displayed on a screen of the provider device 120 and allows a medical provider or trained professional/coach to input commands to control the operation of the application 125. The provider dashboard enables providers to track and manage changes in a patient's metabolic health. The application 125 may be coded as a web page, a series of web pages, or content otherwise coded to render within an internet browser. The application 125 may also be coded as a proprietary application configured to operate on the native operating system of the patient device 110.

The patient health management platform 130 generates various health management insights, for example dynamically generating recommendations for improving a patient's metabolic health based on biological data recorded from a plurality of sources including wearable sensors (or other types of IoT sensors), lab tests, and patient information recorded by the patient. In one embodiment, the patient health management platform 130 predicts a patient's metabolic response based on periodically recorded patient data (e.g., nutrition data, symptom data, lifestyle data). Accordingly, a patient's metabolic response describes a change in metabolic health for the patient resulting from the food they most recently consumed and other aspects of patient data recorded for the patient. Based on the patient's metabolic response, the platform 130 generates a recommendation with instructions for a patient to improve their metabolic health or maintain their metabolic health. Additionally, in real-time or near real-time, the patient health management platform 130 may provide feedback to a patient identifying potential inconsistencies or errors in the food or biological data entered manually by the patient.

The nutrition database 140 stores nutrition data extracted from a collection of nutrient sources, for example food or vitamins. Data within the nutrition database 140 may be populated using data recorded by a combination of public sources and third-party entities such as the USDA, research programs, or affiliated restaurants. The stored data may include, but is not limited to, nutrition information for individual foods or types of foods (e.g., calories, macromolecule measurements, vitamin concentrations, cholesterol measurements, or other facts) and relationships between foods and metabolic responses (e.g., an impact of a given food on insulin sensitivity). Data stored in the nutrition database 140 may be applicable to an entire population (e.g., general nutrition information) or personalized to an individual patient (e.g., a personalized layer of the nutrition database). For example, the nutrition database 140 may store information describing a patient's particular biological response to a food (e.g., the patient's metabolic response to a certain food). In such embodiments, the nutrition database 140 may be updated based on feedback from the patient health management platform 140.

In some embodiments, for example the embodiment illustrated in FIG. 1, the analytics system 100 additionally comprises a research device 150 that analyzes information generated by the patient health management platform. The research device 150 may further evaluate the effectiveness of certain aspects of the treatment recommendation. The research device 150 is a computing device capable of receiving input from a provider with data processing and data communication capabilities. The research device 150 is configured to present a patient's medical history or medically relevant data (i.e., a display screen). The above description of the functionality of the patient device 110 and the provider device 120 also can apply to the research device 150. The research device 150 can be a personal device (e.g., phone, tablet) of the provider, a medical institution computer (e.g., a desktop computer of a hospital or medical facility), etc. In addition, the provider device 150 can include a device that sits within the research office such that a patient can interact with the device inside the office. In such implementations, the research device 150 is a customized device with audio and/or video capabilities (e.g., a microphone for recording, a display screen for text and/or video, an interactive user interface, a network interface, etc.). The research device 150 may also present information to a research team via a mobile application similar to the application described with reference to patient device 110. In one embodiment the research device 150 may conduct precision searches using the patient's own metabolic information to provide a personalized search result related to the patient. As another example, the research device 150 may receive a patient's current metabolic state, their previous metabolic state, and a treatment recommendation that contributed to the current metabolic state. The research device 150 may evaluate the effectiveness of the treatment recommendation as a whole by comparing the patient's current metabolic state and the previous metabolic state, the research device 150 may evaluate the effectiveness of the treatment recommendation as a whole.

Application 155 provides a user interface, herein referred to as a “research dashboard,” that is displayed on a screen of the research device 150 and allows a researcher to input commands to control the operation of the application 155. The research dashboard enables providers to track and manage changes in a patient's metabolic health. The application 155 may be coded as a web page, a series of web pages, or content otherwise coded to render within an internet browser. The application 155 may also be coded as a proprietary application configured to operate on the native operating system of the patient device 110.

Interactions between the patient device 110, the provider device 120, the patient health management platform 130, the nutrition database 140, and the research device 150 are typically performed via the network 160, which enables communication between the patient or user device 120, the provider device 130, and the patient communication platform 130. In one embodiment, the network 160 uses standard communication technologies and/or protocols including, but not limited to, links using technologies such as Ethernet, 802.11, worldwide interoperability for microwave access (WiMAX), 3G, 4G, LTE, digital subscriber line (DSL), asynchronous transfer mode (ATM), InfiniBand, and PCI Express Advanced Switching. The network 160 may also utilize dedicated, custom, or private communication links. The network 160 may comprise any combination of local area and/or wide area networks, using both wired and wireless communication systems.

### Overview of Health Management Platform

The patient health management platform 130 recognizes that a patient's body is a unique system in a unique state in which metabolism is a core biochemical process. Accordingly, insights generated by the patient health management 130 (e.g., the searches conducted, or treatment and nutrition recommendations) are tailored to suit a patient's unique metabolic state and unique parameters or conditions that impact or have previously impacted their metabolic state. As will be described herein, a patient's metabolic health describes the overall effectiveness of their metabolism. For example, a patient's metabolic health may be categorized as impaired, functional, or optimal. The patient health management platform 130 gains insight into a patient's metabolic health by identifying metabolic states occurring over a time period and changes between those metabolic states. The patient health management platform 130 guides a patient towards an improved metabolic state by modeling a patient's physical health over time based on periodically captured biosignals and insights derived from such biosignals. As described herein, a metabolic state represents a patient's state of metabolic health at a specific time (e.g., a state of metabolic health resulting from consumption of a particular food or adherence to a particular medication/treatment). The patent health management platform 130 generates recommendations to improve a patient's metabolic conditions by recommending actions that will optimize biosignal measurements characteristic of a patient's metabolic state. For example, five factors commonly considered include blood sugar, triglycerides, good cholesterol (high-density lipoprotein), blood pressure, and waist circumference.

The patient health management platform 130 determines a current metabolic state of a patient by analyzing a unique combination of continuous biosignals received from various sources including, but not limited to, near-real-time data from wearable sensors (e.g. continuous blood glucose, heart rate, etc.), periodic lab tests (e.g., blood work), nutrition data (e.g., macronutrients, micronutrients, and biota nutrients from food and supplements consumed by the patient), medicine data (e.g., precise dosage and time of medications taken by the patient), and symptom data (e.g., headache, cramps, frequent urination, mood, energy, etc., reported by each patient via a mobile app). The patient health management platform 130 performs this analysis continuously or periodically to establish a time series of metabolic states. As a result, the platform understands not only the current state of each patient, but also the patient's metabolic history (e.g., the patient's history of metabolic states) leading up to the current state. The patient health management platform 130 may conduct patient-specific searches or recommend patient-specific treatments using a patient's current metabolic state and their history of metabolic states. Accordingly, the patient health management platform 130 applies various techniques to establish a personalized metabolic profile for each patient. For example, the patient health management platform 130 may implement a combination of analytic techniques (e.g., analyzing trends, outliers, and anomalies in biosignals as well as correlations across multiple biosignals), rule based artificial intelligence (AI), machine learning-based AI, and automated cohorting or clustering.

For the sake of explanation, the concepts and techniques described herein may be described with reference to diabetes. However, one of skill in the art would recognize that the concepts and techniques may also be applied to any other disease resulting from an impaired metabolism (e.g., metabolic disease). In addition, the concepts and techniques can be applied to other diseases that do not affect a patient's metabolic state, for example autoimmune diseases.

As used herein, the term “continuously” may be used to characterize the collection of biosignals and other data regarding the patient. This term can refer to a rate of collection that is truly continuous (e.g., a constantly recorded value) or near continuous (e.g., collection at every time point or time increment, such as every millisecond, second, or minute), such as biosignals recorded by a wearable device. In some cases, continuously recorded data may refer to particular biosignals that occur semi-regularly, such as a lab test that is taken at a recurring time interval (e.g., every 10 minutes, 30 minutes, hour, 5 hours, day or number of days, week or number of weeks, etc.). The term “continuously” does not exclude situations in which wearable sensors may be removed during certain activities or at times of day (e.g., while showering). In other embodiments, the platform collects multiple biosignals that, in combination, represent a continuous or near continuous signal collection even though some biosignals are collected more frequently than others.

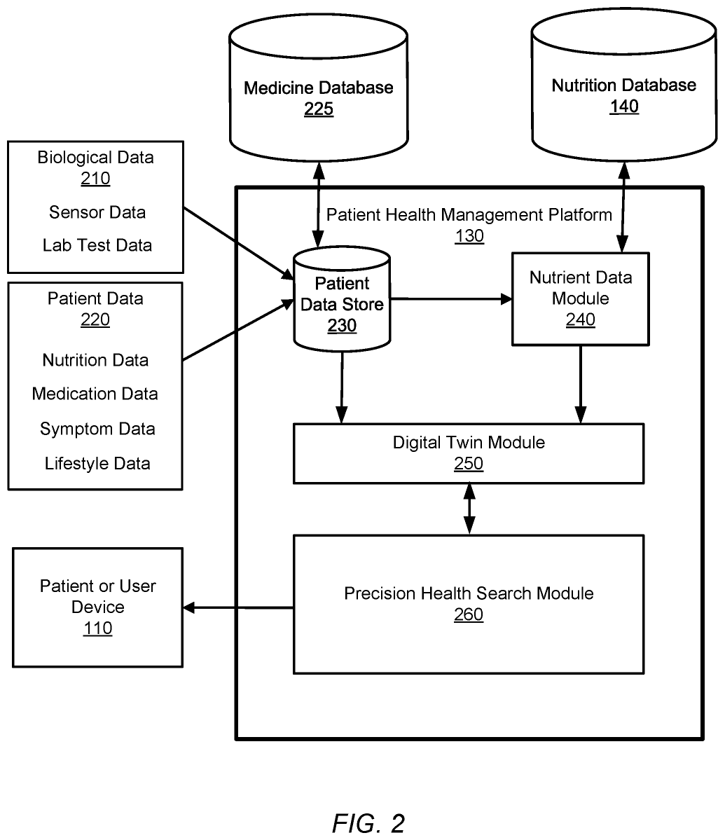

FIG. 2 is a block diagram of the system architecture of the patient health management platform 130 that includes a precision health search module 260, according to one embodiment. The patient health management platform 130 includes a patient data store 230, a nutrient data module 240, a digital twin module 250, and a precision health search module 260. However, in other embodiments, the patient health management platform 130 may include different and/or additional components.

The patient health management platform 130 receives biological data 210 recorded by a variety of technical sources. Biological data 210 includes sensor data comprising biosignals recorded by one or more sensors worn or implemented by a patient. Such biosignals are continuously recorded and each recorded biosignals is assigned a timestamp indicating when it was recorded. Biological data 210 further includes lab test data determined based on blood draw analysis and/or other examinations that a patient has been subjected. Biosignals collected through lab test data may be recorded less frequently than biosignals collected through sensor data, for example over bi-weekly or monthly intervals. In some implementations, lab test data is determined based on procedures and analysis performed manually be doctors or researchers or based on analysis performed by machines and computers separate from the metabolic health manager 100. The patient data store 230 stores biological data 210.

The patient health management platform 130 also receives patient data 220 that is recorded manually by a patient via an application interface on a patient device 110. Patient data 220 includes nutrition data, medication data, symptom data, and lifestyle data. Nutrition data describes a record of foods that a patient has consumed. In some implementations, nutrition data also includes a timestamp indicating when each food was consumed by the patient and a quantity in which each food was consumed. Similarly, medication data describes a record of medications that a patient has taken and, optionally, a timestamp indicating when a patient took each medication and a quantity in which each medication was taken. In response to a patient recording medication data, the patient health management platform may access additional information from a medication database (not shown) to supplement the medication data recorded by the patient. Symptom data describes a record of symptoms experienced by a patient and a timestamp indicating when each symptom was experienced. Lifestyle data describes a record of a patient's physical activity (e.g., exercise) and a record of a patient's sleep history. Lifestyle data may also include a description or selection of emotions or feelings capturing the patient's current state of mind and body (i.e., tired, sore, energetic). In one implementation, each type of patient data 220 may be recorded instantaneously throughout the day when the patient consumes a food, takes a medication, experiences a symptom, or experiences a change in an aspect of their lifestyle. In an alternate implementation, at the end of a day, the patient health management platform 130 detects that a patient has not instantaneously recorded patient data throughout the day and prompts the patient to input a complete record of patient data for the entire day at that time. In addition to biological data 210, the patient data store 230 stores patient data 220.

In some embodiments, the patient data store 230 stores biological data 210 and patient data 230 as an ongoing recorded timeline of entries for a current time period. As new patient data or biological data is recorded or as updates to existing patient data and biological data are received, the patient data store 230 updates the timeline of entries to reflect the new or updated data. Accordingly, the timeline of entries stored in the patient data store 230 comprises foods consumed by the patient at recorded times over the current time period, medications taken by the patient at recorded times over the current time period, and symptoms experienced by the patient at recorded times over the current time period. Some patient data entries may be recorded and reflected in the timeline on a daily basis, whereas other entries are recorded by a patient multiple times a day. Entries for biological data 210, for example, lab test data may be recorded even less frequently, for example as weekly updates to the ongoing timeline. The range of time between a start time and an end time for the current time period may be adjusted manually or trained over time based on predicted and true metabolic states for a patient.

The nutrient data module 240 receives nutrition data from the patient data store 240 and communicates the nutrition data to the nutrition database 140. As described above with reference to FIG. 1, the nutrition database 140 includes comprehensive nutrition information comprising macronutrient information (e.g., protein, fat, carbohydrates), micronutrient information (e.g., Vitamin A, Vitamin B, Vitamin C, sodium, magnesium), and biota nutrients (e.g. Lactococcus, Lactobacillus) for a wide variety of foods and ingredients. In some implementations, the nutrient data module 240 stores nutrition information in a lookup table or combination of lookup tables organized by food item or a category of food item. In other implementations, the nutrient data module 240 stores nutrition information in a lookup table organized by nutrient information or another suitable system. Based on the nutrition data received from the patient data store 230, the nutrient data module 240 identifies nutrition information associated with each food item of the nutrition data and supplements the nutrition data in the patient data store 230 with the identified nutrition information from the nutrition database 140. In some implementations, the nutrient database 140 includes over 100 food-related attributes including, but not limited to, different types of fat, protein, vitamins, and minerals. Nutrition data supplemented by the nutrient data module 240 may be aggregated with the timeline of entries stored in the patient data store 230 to form an aggregated set of patient data for the current time period. The aggregate patient data set may additionally be stored in the patient data store 230, before being input to the digital twin module 250.

The digital twin module 250 generates a digital replica of the patient's metabolic health based on a combination of biological data 210 and patient data 220, hereafter referred to as a digital twin. The digital twin module 250 considers different aspects of a patient's health and well-being to generate and continuously update a patient's digital twin. As described herein, a digital twin is a dynamic digital representation of a patient or of the metabolic function of a patient's human body. The digital twin module 250 continuously monitors biological data and patient data and identifies changes in the patient's metabolic state by correlating a patient's metabolic history with their ongoing medical history. In one embodiment, the digital twin module implements two sets of trained machine-learning metabolic models: a first set of models trained to predict the patient's metabolic state given patient data as inputs to the models and a second set of models trained to determine the patient's true metabolic state given biological data as inputs to the models.

Based on nutrition data, medication data, symptom data, lifestyle data, and supplemental nutrition information retrieved by the nutrient data module 240, the digital twin module 250 generates a prediction of the patient's metabolic state (herein referred to as a patient's “predicted metabolic state”). The digital twin module 250 implements one or more machine-learning, metabolic models to analyze the patient data 220 recorded over a given time period to generate a prediction of the patient's metabolic state for that time period. Accordingly, the prediction of the patient's metabolic state is a function of a large number of metabolic factors recorded in the patient data 220 (e.g., fasting blood glucose, sleep, and exercise) and a nutrition profile (e.g., macronutrients, micronutrients, biota nutrients).

In one embodiment, the digital twin module 250 includes several machine-learning metabolic models, such that each metabolic model is trained to predict an effect of a single aspect of the patient data 220. For example, a first model may be trained to predict an impact of a patient's symptoms on their metabolic state, a second model may be trained to predict an impact of various lifestyle choices (e.g., sleep and exercise habits) on the patient's metabolic state, and a third model may be trained to predict on impact of nutrition data recorded for a time period on the patient's metabolic state. The digital twin module 250 aggregates the output of each metabolic model to determine a holistic representation of patient's metabolic state that characterizes the combined effect of the patient's symptoms, lifestyle choices, and nutrition on their metabolic state.

As described herein, metabolic models used to predict a patient's biological response (e.g., a change in their metabolic state) are trained using a training data set comprised of labeled metabolic states and a record of patient data 220 that contributed to each labeled metabolic state. During the training process, the metabolic model determines the impact of a biosignal(s) or an aspect of patient data (e.g., particular types of foods, medications, symptoms, or lifestyle adjustments) on a patient's metabolic state by drawing correlations and relationships between recorded patient data and each labeled metabolic state, for example the impact of given foods or medications on insulin sensitivity. Once trained, the metabolic model predicts a patient's metabolic state given an aspect of the patient data 220 as an input(s). By aggregating the output of each metabolic model, the digital twin module 250 generates a predicted change in patient's metabolic state resulting from a complete set of patient data inputs by aggregating the output of each metabolic model.

In addition to the predicted metabolic state, the digital twin module 250 may implement one or more metabolic models to generate a true representation of a patient's metabolic state (herein referred to as a “true metabolic state”) based on the biological data 210 recorded for a time period. In comparison to the metabolic models used to generate a prediction of a patient's metabolic model, the digital twin module 250 trains metabolic models implemented by the digital twin module 250 to determine the true metabolic state of the patient to process aspects of biological data 210 (e.g., wearable sensor data and lab test data) into an effect on the patient's metabolic state. For such implementations, at the conclusion of a time period, a metabolic model may be trained to analyze biological data 210 recorded by wearable sensors during the time period and determined based on lab tests from the time period to determine a true metabolic state for the patient that reflects the actual biological conditions experienced by a patient (e.g., their HbA1c levels, or BMI) during the time period. Accordingly, given biological data 210 as an input, the metabolic model is further trained to output a patient's actual biological response (e.g., a measured insulin sensitivity or change in glucose in response to consuming a food or taking a medication).

In one embodiment, the digital twin module 250 includes several machine-learning metabolic models, such that each metabolic model is trained to predict an effect of a single aspect of the biological data 220. For example, a first model may be trained to determine a change in a patient's true metabolic state based on measurements of their HbA1c levels and a second model may be trained to determine a change in a patient's true metabolic state based on BMI measurements. The digital twin module 250 aggregates the output of each metabolic model to determine a holistic representation of the patient's true metabolic state that characterizes the combined effect of the HbA1c levels and the BMI measurements.

As described herein, metabolic models applied by the digital twin module 250 to output a patient's true metabolic state are trained using a training data set comprised of labeled metabolic states and a record of biological data 210 that contributed to each labeled metabolic state. During the training process, a metabolic models determines an actual impact of an aspect of patient data 220 or biological data 210 (e.g., particular types of foods, medications, symptoms, or lifestyle adjustments) by drawing correlations and relationships between aspects of biological data 210 and a corresponding labeled metabolic state. Once trained, the metabolic model generates a true representation of a patient's metabolic state given an aspect of the biological data 210 as an input. By aggregating the output of each metabolic model, the digital twin module 250 determines of a true change in patient's metabolic state resulting from a complete set of biological data inputs by aggregating the output of each metabolic model.

Patient data and biological data may be recorded at varying intervals. For example, sensor data is recorded continuously every 15 minutes, lab test data is recorded bi-weekly, and patient data 220 is recorded multiple times a day as needed. Therefore, the patient health management platform 130 may not receive an updated recording for every type of data in time to generate a predicted metabolic state. When generating a predicted metabolic state for a particular time period, the digital twin module 250 retrieves all patient data 220 recorded within that time period and the metabolic state predicted by the during the preceding time period. In some embodiments, the digital twin module 250 implements one or more machine learning models to process, as inputs, the recorded patient data and the most recently predicted metabolic state into a predicted metabolic state for a current time period. In place of the most recent predicted metabolic state, the digital twin module 250 may input the most recent true metabolic state to the one or more machine learning models. Accordingly, the predicted metabolic state reflects any effects that the most recently recorded patient data 220 had on a previous metabolic state.

Similarly, when generating a true metabolic state for a time period, the digital twin module 250 retrieves all biological data 210 recorded within that time period (e.g., heart rate, exercise, continuous blood glucose, ketones, blood pressure, weight) and the true metabolic state generated during the preceding time period. The digital twin module 250 may also rely on one or more machine learning models to process the retrieved biological data 210 and the most recent true metabolic state into a current true metabolic state. Accordingly, the generated true metabolic state also reflects any effects of the most recently recorded biological data 210 had on a previous metabolic state. For example, a machine learned model may use a continuous blood glucose signal measured every 15 minutes to calculate a patient's 5-day average blood glucose. The computed measurement is compared against established ranges in the medical literature to determine whether the patients are in a diabetic, pre-diabetic, or non-diabetic state as they progress with their treatment. In common implementations, the digital twin module 250 updates a patient's metabolic state at a higher frequency than a frequency at which lab test data is recorded. As such, when lab test data is unavailable for the current time period, the digital twin module 250 may generate the updated metabolic state based on the lab test data recorded most recently for a preceding time period.

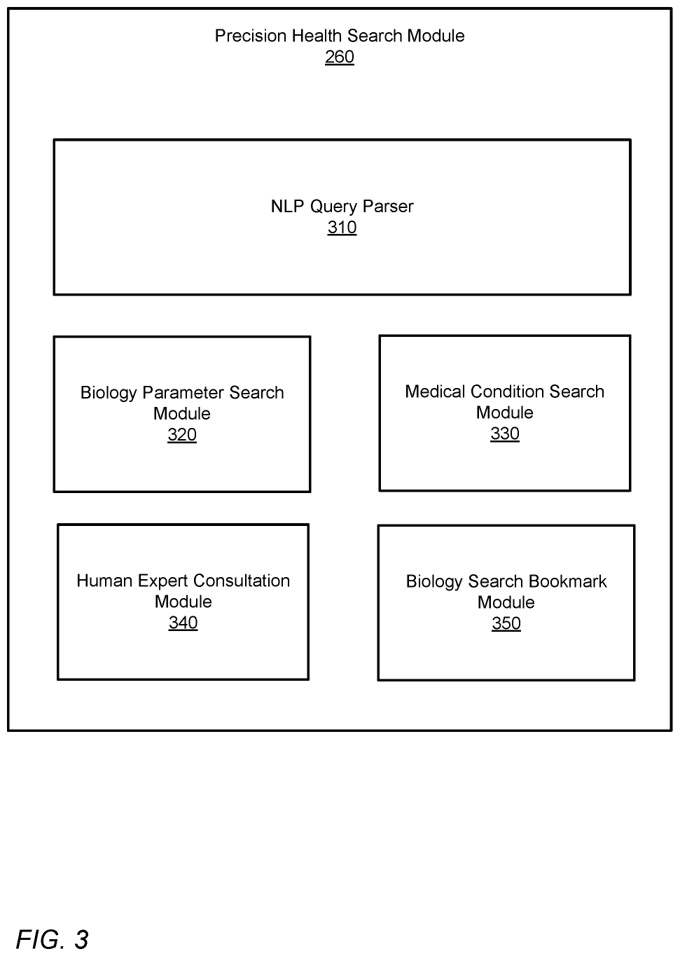

The patient health management platform 130 may include a precision health search module 260 that intelligently searches biological information, such as biomarkers and medical conditions of an individual, based on the digital twin module 250. A patient's metabolism can be measured using hundreds of biomarkers, so querying results about a specific aspect of their metabolic health can be a difficult and time-consuming process. The precision health search module 260 automates the process of identifying relevant health components for a given individual and presenting the relevant health components to the patient in a user-friendly visualization. Accordingly, the precision health search module 260 enables a patient to gain a unique insights into the patients' metabolic health and enables clinicians to improve patient care based on patient-specific insights. The precision health search module 260 may perform biomarker searches, which search elements of an individual's digital twin module 250 related to particular health parameters. The precision health search module 260 may further perform medical condition searches, which returns a patient-specific risk of a given medical condition. The precision health search module 260, biomarker searches, and medical condition searches are described in greater detail below with regards to FIG. 3. A person of ordinary skill in the art would appreciate that the examples described herein are illustrative and the precision health search module 260 may perform other types of searches including, but not limited to, searches related to any biomarkers or any biosignal data collected for a patient.

### Precision Health Search Module

FIG. 3 illustrates a block diagram of a precision health search module, according to one embodiment. In the illustrated embodiment of FIG. 3, the precision health search module 260 includes a natural language processing (NLP) query parser 310 (or any other suitable query parser), a biology parameter search module 320, a medical condition search module 330, a human expert consultation module 340, and a biology search bookmark module 350. However, in other embodiments, the patient health management platform 130 may include different and/or additional components.

The NLP query parser 310 may process user inputs (e.g., search queries) and the processed inputs may be used for subsequent search in the digital twin module 250. The NLP query parser 310 receives a search query from a user requesting a search for biological information specific to a patient and processes user inputs into a machine-interpretable format. In one embodiment, the user input is textual data, which may be free form or structured. For example, a user may input unstructured text, such as the word “potassium,” or “sodium.” As another example, the user may input a question or a piece of text, such as “Do I have fatty liver?” or “What is my risk of getting fatty liver?” The NLP parser 310 generates tokens corresponding to the user input. In one embodiment, the NLP parser 310 parses the search query into a string of tokens by tokenizing the user input using any available NLP machine learning model(s). Continuing from the “Do I have fatty liver?” example query, the NLP parser 3100 may generate tokens including “do”, “I,” “have,” and “fatty liver” (a medical condition).

In one embodiment, the NLP query parser 310 classifies the search query into a search type, for example a biological parameter search that searches for an health component related to biomarkers or a medical condition search that searches for a medical condition and returns a user-specific risk for the medical condition. The NLP query parser 310 may perform a supervised classification or an unsupervised classification. In embodiments where the NLP query processor 310 performs a supervised classification, the NLP query parser 310 classifies the user inputs into a predetermined category, such as a biological parameter search or a medical condition search. In some embodiments, the precision health search module 260 does not input a user's search string to the NLP query parser 310 before conducting the search.

In some embodiments, the NLP query parser 310 further determines an intent associated with the user input based on the tokenized input data. In one embodiment, the NLP query parser 310 determines the intent of the user by applying a machine-learning model trained using a training dataset of example queries labeled with the corresponding intent of the user. For example, the NLP query parser 310 may determine that the intent for the query “my risk of getting fatty liver” is to obtain the probability that a given person will develop a fatty liver condition. The NLP query parser 310 may input the determined intent to the biology parameter search module 320 or the medical condition search module 330 depending on the classification of the user input. In some embodiments, the NLP query parser 310 is not included and the determination of intent associated with the user input does not occur.

The biology parameter search module 320 searches a biological parameter ontology for the input query itself or for tokens generated from the input query and returns health parameters, for example biomarkers and nutrients, associated with the input query or the input token. The biology parameter search module 320 may search the entered query or tokens generated from the input query (e.g., “potassium”) in a biological parameter ontology. As described herein, a biological parameter ontology is a knowledge model, which includes a set of concepts within a particular domain and models the relationships between the set of concepts. As described below, a biological parameter ontology is a hierarchical data structure organized into one or more levels in order of increasing specificity. A node at a higher level (referred to herein as a “primary node”) represents a broader term or category than nodes at a lower level connected to the primary node (referred to herein as “secondary nodes”).

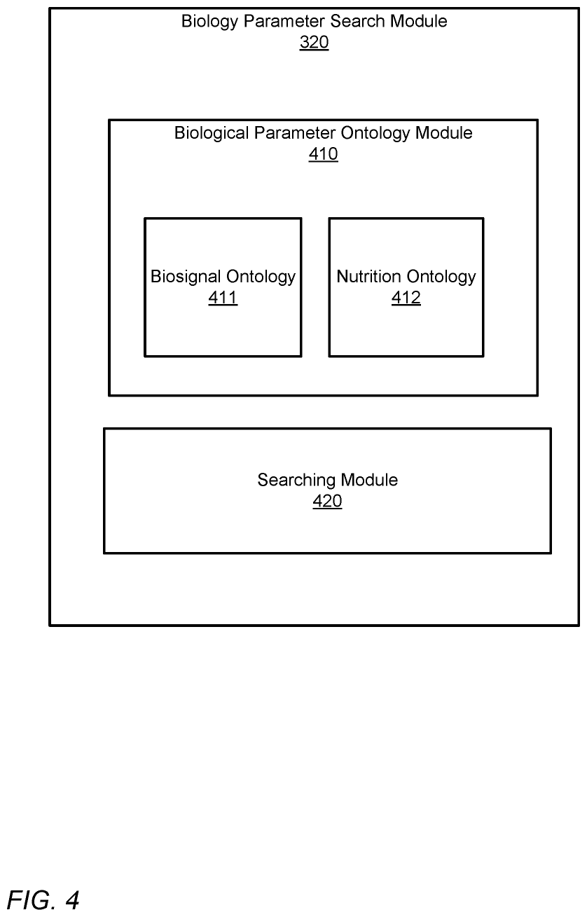

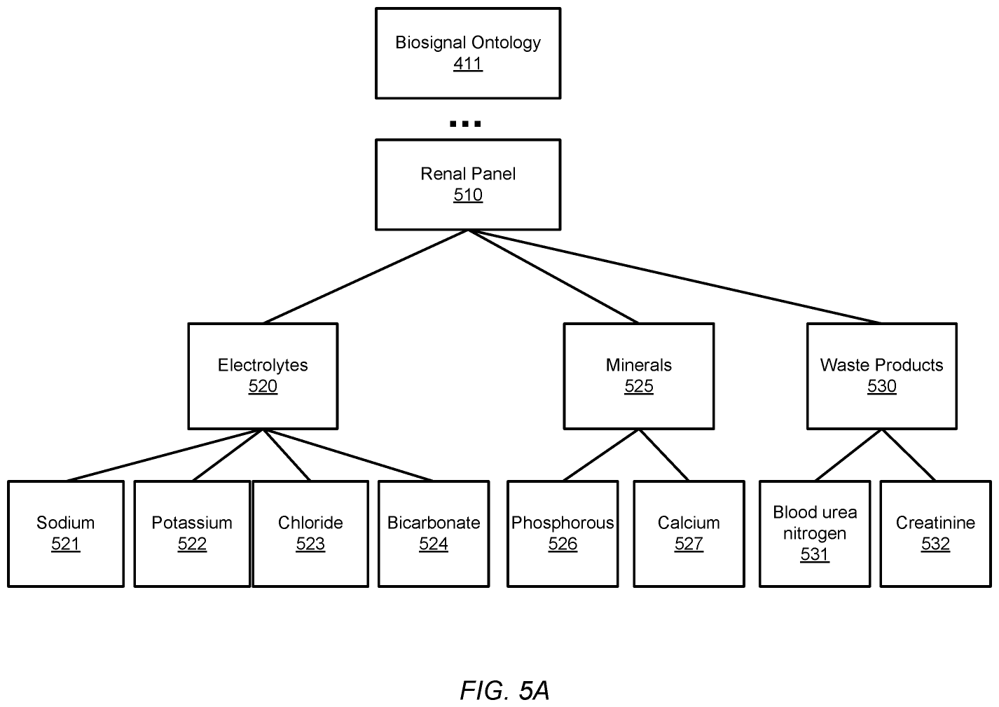

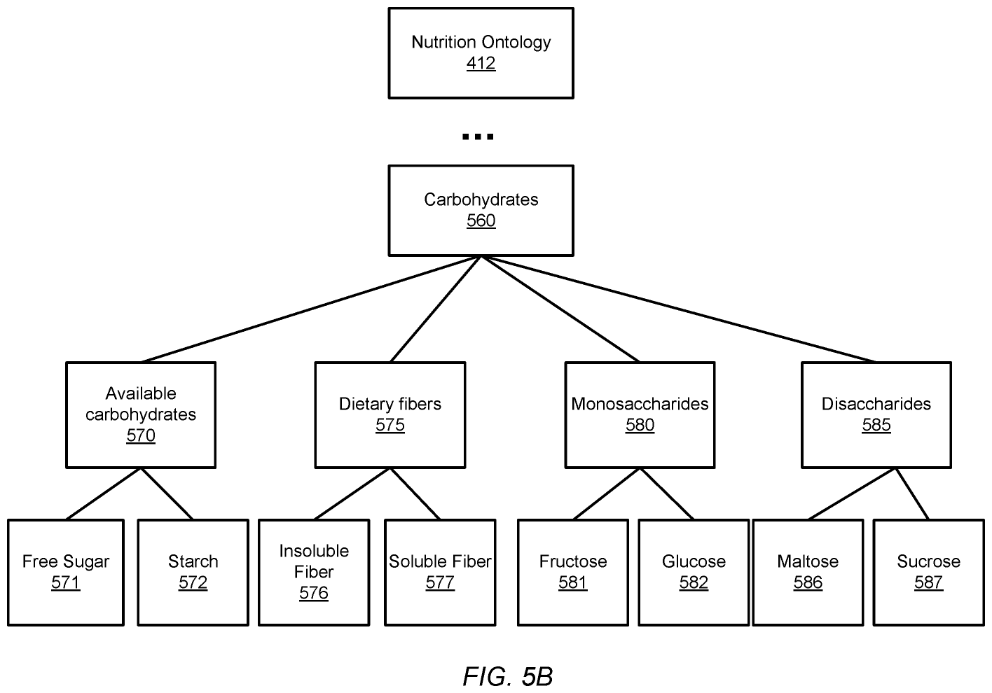

Each ontology described herein may further be separated into one or more sub-ontologies. The biology parameter search module 320 searches one or more biological parameter ontologies and returns biomarkers or nutrient information related to the search query. In some embodiments, the precision health search module 320 displays the results of the search in a user interface, for example the interface illustrated in FIG. 5. The biology parameter search module 320 is further discussed below with reference to FIGS. 4-5.

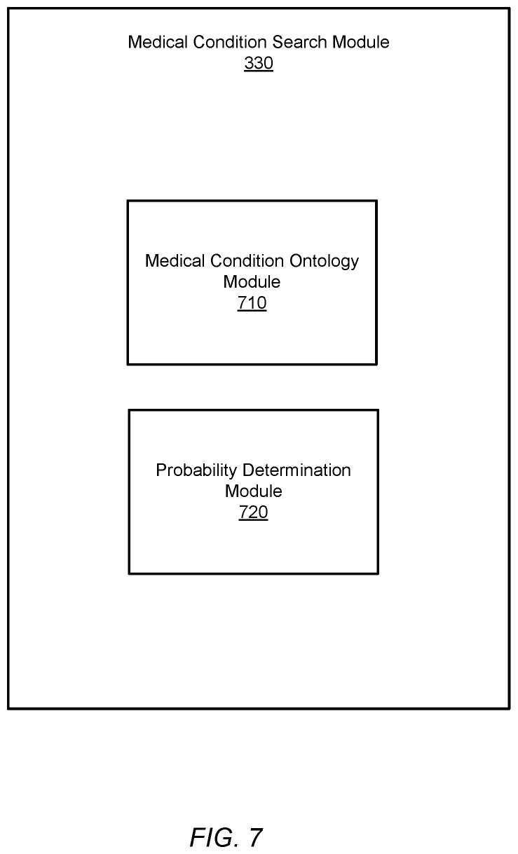

The medical condition search module 330 may search and return a personalized likelihood, (also referred to herein as a patient-specific risk score) that a person will be affected by one or more medical conditions based on the person's metabolic health modeled by the digital twin module 250. The medical condition search module 330 searches a machine-interpretable query (such as user inputs stating “probability of developing a Fatty Liver condition”) in a metabolic condition ontology. For example, the medical condition search module 330 processes medical condition searches such as “Do I have fatty liver?” and “What is my risk of getting fatty liver?” The medical condition search module 330 locates “fatty liver” in a medical condition ontology and returns various means for evaluating or determining whether a person has or will contract Nonalcoholic fatty liver disease (NAFLD). The medical condition search module 330 may further retrieve, from the digital twin module 250, values for one or more biosignal measurements to be used in the determination. The medical condition search module 330 is further discussed below with reference to FIG. 7.

The human expert consultation module 340 provides a user with an option to consult human expert (e.g., a medical professional). When the biology parameter search module 320 or the medical condition search module 330 do not provide a relevant answer or when a user requests additional information, the human expert consultation module 340 may offer the user an option to communicate with a human expert with skill or experience relevant to the user's search query. In one embodiment, the human expert consultation module 340 may offer the user an option to pay for the answer or advice.

The biology search bookmark module 350 provides an option for a user to bookmark the results returned by the biology parameter search module 320 or the medical condition search module 330. The biology search bookmark module 350 may also provide an option for a user to share the bookmarked results with others, for example other members of a clinical care team (e.g., other doctors, clinicians, or nurses), family members of a patient, etc. In some embodiments, when a user requests to bookmark the search results, the biology search bookmark module 350 saves the user's initial search query with any tokenized representation of the search query and the search results returned for the given person in a database of search results (not shown).

FIG. 4 illustrates a block diagram of a biology parameter search module 320, according to one embodiment. In the illustrated embodiment of FIG. 4, the biology parameter search module 320 includes a biological parameter ontology module 410 and a search module 420. The biology parameter search module 320 further includes a biomarker subtree 411 and a nutrition ontology 412. However, in other embodiments, the biology parameter search module 320 may include different and/or additional components.

The biological parameter ontology module 410 includes one or more hierarchical data structures for biology parameters (or biomarkers), for example the biosignal ontology 411 and the nutrition ontology 412. The biological parameter ontology module 410 organizes biological concepts including biomarkers (e.g., “blood potassium”, “blood glucose”, and “resting heart rate”) and nutrients (e.g., “dietary potassium”, “dietary magnesium”, and “dietary sodium”) into a searchable data structure. By traversing the structure of the ontology, the biological parameter ontology module 410 determines other terms related to a query (e.g., “potassium”) beyond the measurements of a biomarker specified in the query (e.g., levels of potassium). In the illustrated embodiment, the biological parameter ontology module 410 includes two separate ontologies—one biosignal ontology 411 and one nutrition ontology 412, which are further described below. Each of the ontologies 411 and 412 may further be divided into separate sub-ontologies related to biosignals and/or nutrition.

The biological parameter ontology module 410 maps each concept in the hierarchical data structure to a specific component (e.g., biomarker) stored within the digital twin module 250. Accordingly, the biological parameter ontology module 410 maps each node of the ontology data structure to one or more biomarkers stored within the health profile of the patient. In one embodiment, the biological parameter ontology module 410 maps each node of the biosignal ontology 411 and/or the nutrition ontology 412 to one or more biomarkers of the digital twin module 250. By mapping health parameters in the data structure to biomarkers within the digital twin module 250, the biological parameter ontology module 410 may query the digital twin module 250 to return health parameters relevant to the query. In the illustrated embodiment of FIG. 4, the biological parameter ontology module 410 may include one or more biosignal ontology 411 and one or more nutrition ontology 412.

A biosignal ontology 411 includes health parameters correlated with signals extracted from actual biomarker measurements. For example, a biosignal ontology 411 may describe bloodwork diagnostics. In one embodiment, the searching module 420 searches the biosignal ontology 411 to identify one or more biosignal parameters related to the search query. As described above, the biosignal ontology 411 maps each identified health parameter to one or more biomarkers characterized by biosignal measurements. Accordingly, the searching module 420 accesses the biosignal measurements collected for the given patients using the techniques described above with reference to FIGS. 1 and 2.

One such ontology 411 may include a sublevel describing parameters collected from a renal panel, which may be used to characterize kidney function (e.g., a high blood urea nitrogen level may indicate possible kidney damage). FIG. 5A illustrates an example set of branches within the biosignal ontology 411, according to one embodiment. Each horizontal row of boxes represent a level of the partial ontology 500. Each box illustrated in the partial ontology 500 represents a node of the partial ontology 500 and each node represents either a category of biosignal parameters or a specific biosignal parameter depending on the level of the node. For example, the biosignal ontology 411 may include a level 510 (e.g., a primary node) representing a renal panel and measurements taken for patents during a renal panel. Within the renal panel level 510, there are different categories of biosignals (e.g., secondary nodes) collected during a renal panel. In the illustrated ontology 500, the renal panel level 510 include a sublevel with secondary nodes such as electrolytes 520, minerals 525, and waste products 530. The sublevel for measured electrolytes 520 branches into a further sublevel including electrolytes in the blood such as sodium 521, potassium 522, chloride 523, and bicarbonate 524. The sublevel for measured minerals 525 branches into a further sublevel including minerals in the blood such as phosphorous 526 and calcium 527. The sublevel for waste products 530 includes into a further sublevel including blood urea nitrogen 531 and creatinine 532. In the illustrated partial ontology 500, renal panel 510 represents the primary node that branches into more granular sublevels of the biosignal ontology 411 (e.g., electrolytes 520, minerals 525, and waste products 530).

The nutrition ontology 412 includes health parameters extracted from a patient's nutrient intake data (e.g. nutrition data recorded by the patient). In one embodiment, the searching module 420 searches the nutrition ontology 412 to identify one or more health parameters related to the search query. As described above, the nutrition ontology 412 maps each identified health parameter to one or more biomarkers characterized by nutrition information. Accordingly, the searching module 420 accesses the biosignal measurements predicted for the given patients based on nutrition information and using the techniques described above with reference to FIGS. 1 and 2.

FIG. 5B illustrates an example set of branches within the nutrition ontology 412, according to one embodiment. Each horizontal row of boxes represents a level of the partial tree 550. Each box illustrated in the partial ontology 550 represents a node of the partial ontology 550 and each node represents either a category of health parameters or a specific health parameter depending on the level of the node. For example, the nutrition ontology 412 may include a level 560 (e.g., a primary node) representing carbohydrates that ingested by patients as part of their meals. Within the carbohydrate level 560, each subtype of carbohydrate (e.g., secondary nodes) affects the metabolic health of a patient differently. For example, soluble fiber has prebiotic properties that are beneficial to gut microbiota. In comparison, excessive consumption of sucrose can cause increases in a patient's blood glucose, causing metabolic harm and result in diabetes. In the illustrated ontology 550, the carbohydrate level includes a sublevel with secondary nodes such as available carbohydrate 570, dietary fiber 575, monosaccharide 580, and disaccharide 585. The sublevel for available carbohydrate 570 branches into a further sublevel including carbohydrates such as free sugar 571 and starch 572. The sublevel for dietary fibers 575 branches into a further sublevel including insoluble fibers 576 and soluble fibers 577. The sublevel for monosaccharides 540 branches into a further sublevel including fructose 581 and glucose 582. The sublevel for disaccharides 550 branches into a further sublevel including maltose 586 and sucrose 587. In the illustrated partial ontology 500, carbohydrates 510 represents the primary node that branches into more granular sublevels of the nutrition ontology 412 (e.g., available carbohydrates 570, dietary fibers 575, monosaccharides 580, and disaccharides 585).

Returning to FIG. 4, the searching module 420 searches biological parameter ontologies stored in the biological parameter ontology module 410 to generate a response to a given query or tokenized representation of the query. The searching module 420 receives a search query and/or identifies search terms in a query (i.e., “potassium.”). The searching module 420 searches a biological parameter ontology (e.g., the biosignal ontology 411 and the nutrition ontology 412) to identify text matches to the query or search terms of the query (e.g., the word “potassium”). Based on the text matches, the searching module 420 identifies one or more health parameters related to the search query.

For example, in response to the search term “potassium,” the searching module 420 may identify two matches: 1) “blood potassium” located in the Electrolyte Panel sub-level of a biosignal ontology 411 and 2) “dietary potassium” located in the Micronutrient sublevel of the nutrition ontology 412. The searching module 420 traverses the nodes stemming from the “blood potassium” primary node of the biosignal ontology 411 and identifies the secondary nodes representing “hypokalemia” and “hyperkalemia” in the medical condition sub-level branching from “blood potassium.” Hypokalemia is a medical condition characterized by excessively low blood potassium and hyperkalemia is a condition characterized by excessively high blood potassium. Because both hypokalemia and hyperkalemia are medical conditions linked to the “blood potassium” node, the searching module 420 identifies both conditions as related to the user's initial search. Similarly, the searching module 420 traverses the nodes stemming from the identified “dietary potassium” primary node in the nutrition ontology 412 and identifies the secondary node representing “hypertension” in the medical condition sub-level branching from “dietary potassium.” Hypertension is a medical condition characterized by high blood pressure, which may be caused by insufficient dietary potassium. Because hypertension is a medical condition linked to the “dietary potassium” node, the searching module 420 identifies the condition as related to the user's initial search.

In some embodiments, the search results collected by the searching module 420 include measurable health parameters, for example the blood potassium and dietary potassium. In such embodiments, searching module 420 accesses patient-specific measurements collected for the biomarkers correlated with the health parameters identified in the search results as described above. In some embodiments, the searching module 420 accesses a single biomarker measurement for a given health parameter (e.g., blood potassium). In other embodiments, the searching module 420 accesses multiple biomarker measurements to characterize a given health parameter. The digital twin module 250 determines biomarker measurements using the techniques described above with reference to FIG. 2, for example by applying machine-learning models to predict a measurement based on patient-provided nutrition information or biosignals collected for the patient.

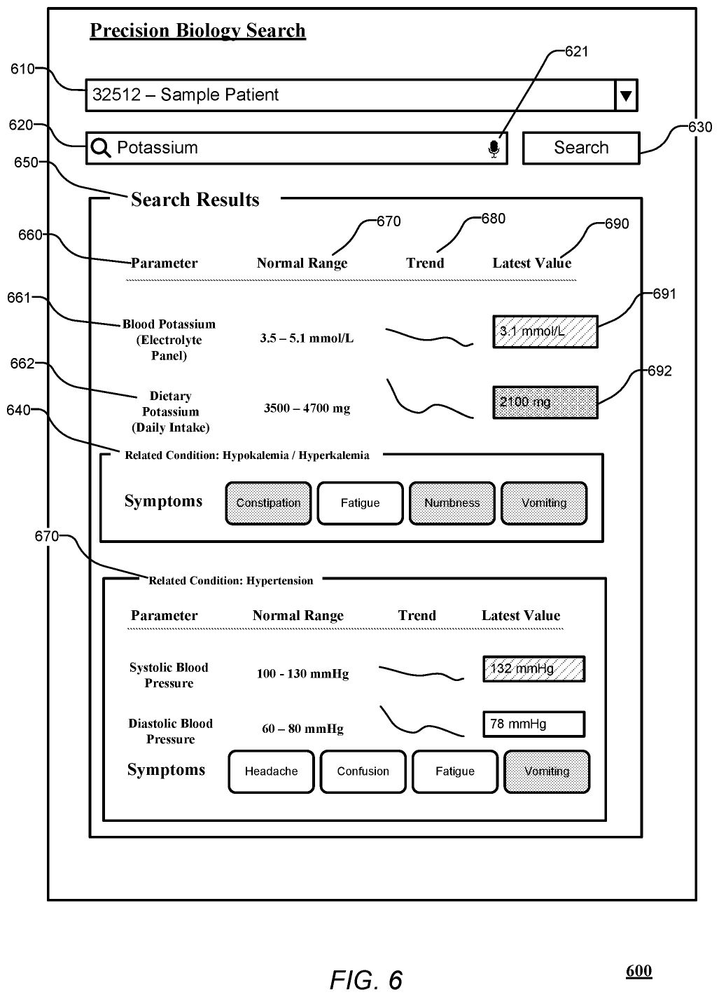

The searching module 420 may display the search results (e.g., health parameters identified by the searching module 420) to a user via a user interface, for example the interface illustrated in FIG. 6. FIG. 6 illustrates an example user interface displaying the results of a biology parameter search, according to one embodiment. The user interface 600 displays the results of the search generated by the searching module 420 to the user. The searching module 420 renders a graphical user interface that includes a graphical element corresponding to each health parameter included in the search results. The graphical element may include measurements collected or prediction by the digital twin module 250 for a given patient. The illustrated interface 600 includes an identification bar 610 where a user can select or supply an identifier associated with a patient. In one embodiment, the identifier is a unique number assigned to the patient. Alternatively, the identifier may be the patient's name, social security, or any other information that can be used to identify a patient. The illustrated interface 600 further includes a search bar 620 where a user may input a search query. In some embodiments, the search query is a sequence of input terms. In other embodiments, the search query is a question. For example, the user may input the term “potassium” in the search bar 620 or the question “what are the risks of low potassium?” In some embodiments, the search bar 620 includes a button 620 that allows a user to supply a search query using voice recognition techniques that convert audio messages into digital messages. After supplying a search query to the search bar 620, a user may execute the search by selecting the search button 630.

After completing the search of the biosignal ontology 411 and the nutrition ontology 412, the searching module 420 updates the user interface 600 to display the search results 650. In some embodiments, the search results include measurable health parameters 660, for example blood potassium and dietary potassium. For each health parameter 660, the search results 650 also display a normal range of measurements 670 corresponding to the health parameter, a trend 680 in the measurements for the health parameter recorded for the identified patient, and the latest value 690 of the health parameter recorded for the identified patient. In the illustrated embodiment, the user interface 600 displays the trend 680 as a graph of the patient's historical measurement for biomarkers associated with the health parameter, for example historical measurements collected from wearable sensors, lab test, user inputs, or predictions generated by the digital twin module 250. More information regarding patient-specific biomarker predictions can also be found in U.S. Pat. No. 11,707,226, filed on Mar. 16, 2021, which is incorporated by reference herein in its entirety. In some embodiments, user interface displays the latest values 690 with different visual representations to indicate whether the patient's latest measurement falls within a range of healthy measurements, a range of measurements indicating the patient is at-risk, or a range of unhealth measurements. The illustrated interface 650 displays two health parameters related to the search query “potassium”—blood potassium 661 and dietary potassium 662.

The user interface 600 may further display health parameters identifying medical conditions related to the user's search query and relevant symptoms for each search query. For example, the illustrated interface 600 displays the related medical conditions 640 hypokalemia and hyperkalemia (e.g., the medical conditions linked to the Blood Potassium node in the biosignal ontology 411 described above). The interface 600 also presents typical symptoms of both conditions including constipation 641, fatigue 642, numbness 643, and vomiting 644. The interface 600 also displays the related medical condition 670 hypertension (e.g., the medical condition linked to the Dietary Potassium node in the nutrition ontology 412 described above). The interface 600 also presents typical symptoms of hypertension including headache, confusion, fatigue, and vomiting. In some embodiments, the user searching module 420 may update the user interface to display symptoms that the patient has already exhibited in a visually different representation, for example a darker background as illustrated in the interface 600. Accordingly, a user may visualize whether a patient has begun exhibiting a many of the symptoms related to the medical condition.

FIG. 7 illustrates a block diagram of a medical condition search module 330, according to one embodiment. In the illustrated embodiment of FIG. 7, the medical condition search module 330 includes a medical condition ontology module 710 and a probability determination module 720. However, in other embodiments, the medical condition search module 330 may include different and/or additional components.

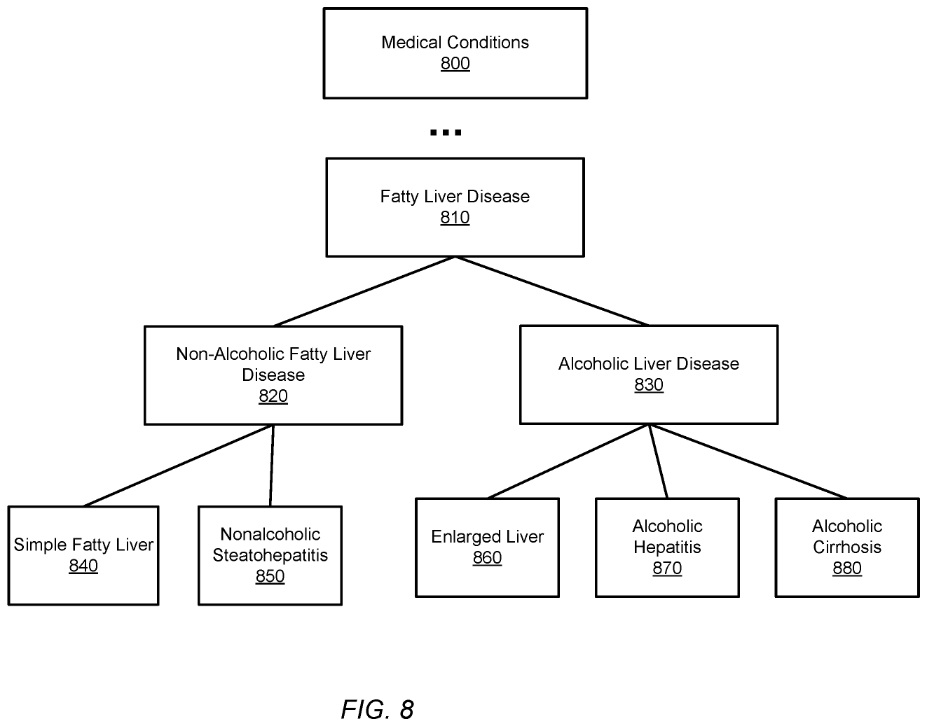

The medical condition ontology module 410 includes a hierarchical data structure that includes a group of medical conditions and their relationships to each other. The hierarchical data structure is referred to herein as a “medical condition ontology.” In some embodiments, the medical condition ontology module 710 stores and applies medical industry standard ontologies, for example SNOMED CT and ICD-11. Given the structure of the of the ontology, the medical condition ontology module 710 determines medical conditions related to a query. For example, given an input query of “fatty liver,” the medical condition ontology module 710 may organize the multiple types and subtypes of Fatty Liver diseases in the hierarchy, for example as shown in FIG. 8. In one embodiment, the searching module 420 searches the biosignal ontology 411 to identify one or more health parameters related to the search query. In one embodiment, the medical condition ontology module 410 stores a signal medical condition ontology that branches into several more specific sub-ontologies. In other embodiments, the medical condition ontology module 410 stores multiple medical condition ontologies.

FIG. 8 illustrates an example set of branches within the medical condition ontology including multiple sublevels and nodes, according to one embodiment. As described above with reference to FIG. 5, each box illustrated into the medical conditions ontology 800 represents a node of the partial tree 800 and each node represents either a category of medical conditions or a specific medical condition. Each horizontal row of boxes represents a level of the ontology 800 such that deeper levels of the ontology 800 represent more specific characterizations of medical conditions. For example, the nodes stemming from Fatty Liver Disease 810 may be a partial tree within the ontology 800. The Fatty Liver Disease sublevel branches into two more specific nodes—non-alcoholic fatty liver disease 820 and alcoholic liver disease 830. The node for non-alcoholic fatty liver disease 820 further branches into specific medical conditions simple fatty liver 840 and nonalcoholic steatohepatitis 850, both of which are particular non-alcoholic fatty liver diseases. The node for alcoholic liver disease 830 further branches into specific medical conditions: enlarged liver 860, alcoholic hepatitis 870, and alcoholic cirrhosis 880, each of which is a particular alcoholic fatty liver disease.

Another set of branches within the medical condition ontology (not shown) may characterize hypertensive diseases. For example, the primary node for hypertensive diseases may branch into secondary nodes describing categories of hypertensive diseases (e.g., essential hypertension, hypertensive heart disease, hypertensive renal disease, and secondary hypertension). Each category may further branch into particular hypertensive diseases within the category. For example, the secondary node for essential hypertension may branch into particular hypertensive diseases (e.g., combined diastolic and systolic hypertension, isolated diastolic hypertension, isolated systolic hypertension). The secondary node for secondary hypertension may further branch into particular diseases (e.g., congenital renal artery stenosis, hyperaldosteronism, and pre-existing hypertension complicating pregnancy).

Returning to FIG. 7, the probability determination module 720 employs the techniques described above with reference to the searching module 420 to search medical condition ontologies stored in the medical condition ontology module 710 in response to a given query or tokenized representation of the query. The probability determination module 720 receives a search query and/or identifies search terms in a query (i.e., “fatty liver disease.”). The probability determination module 720 searches a medical condition ontology to identify text matches to the query or search terms of the query (e.g., the phrase “fatty liver disease”). Based on the text matches, the probability determination module 720 identifies one or more medical conditions related to the search query. In one embodiment, the probability determination module 720 treats each node identified during the text matching as a primary node and identifies medical conditions related to the search query by traversing the medical condition ontology 710 through secondary nodes connected to each primary node.

The probability determination module 720 determines a patient-specific risk assessment for one or more medical conditions. The probability determination module 720 combines biomarkers measurements and patient data retrieved from the digital twin module 250 to determine a probability that a given patient will be affected by one or more medical conditions (e.g., medical conditions related to a fatty liver disease 810). As described herein, the patient-specific risk score for a medical condition describes a likelihood that a patient is or will be affected by the medical condition.

In some embodiments, the probability determination module 720 inputs one or more biomarker measurements to a machine-learning model(s) trained to predict patient-specific risk scores one or more of the identified medical conditions. In some embodiments, the probability determination module 720 determines a patient-specific risk score for each medical condition in the medical condition ontology based on a range of measurements personalized for the patient. For example, a healthy range of measurements for a given health parameter may vary amongst different patients depending on various demographic parameters (e.g., age and gender) and other health parameters. In one embodiment, the probability determination module 720 determines the patient-specific risk score for a medical condition by comparing biomarker measurement(s) collected/predicted for a patient to a range of biomarker measurements collected for a healthy individual. For example, the farther a biomarker measurement is from the health range, the higher the patient-specific risk score associated with that medical condition. In some embodiments, the probability determination module 720 inputs biomarker measurements for a patient to a machine-learning model trained based on a training dataset of biomarker measurements and medical conditions labeled with an actual patient-specific risk score.

Returning to the ontology illustrated in FIG. 8, the probability determination module 720 may determine a patient-specific risk score for each of the medical conditions Simple Fatty Liver 840, NASH (Nonalcoholic Steatohepatitis) 850, Enlarged Liver 860, Alcoholic Hepatitis 870, and Alcoholic Cirrhosis 880. In some embodiments, the probability determination module 720 may aggregate the risk score determined for each medical condition into an aggregate risk that the patient will be affected by the more general category of medical conditions (represented by a parent node). For example, the probability determination module 720 may aggregate the patient-specific risk scores determined for simple fatty livers 840 and nonalcoholic steatohepatitis 850 to determine a patient-specific risk score for non-alcoholic fatty liver disease. Similarly, the probability determination module 720 may aggregate the patient-specific risk scores determined for enlarged liver 860, alcoholic hepatitis 870, and alcoholic cirrhosis 880 to determine a patient-specific risk score for alcoholic liver disease 830. The probability determination module 720 may further aggregate the patient-specific risk scores determined for non-alcoholic fatty liver disease 820 and alcoholic liver disease 830 to determine a patient-specific risk score for fatty liver disease 810.

In embodiments where the precision health search module 260 performs a medical condition search (not shown), the probability determination module 720 may update the graphical user interface illustrated in FIG. 6 to display the searched medical conditions and a likelihood that a patient has the searched medical condition. In some embodiments, the probability determination module 720 generates a patient-specific treatment recommendation outlining objectives for patient to complete to improve their health given the patient-specific risk scores. More information regarding patient-specific treatment recommendations can be found in U.S. Pat. No. 11,707,226, filed on Mar. 16, 2021, which is incorporated by reference herein in its entirety.

In some embodiments, the medical condition ontology module 710 comprises an additional medical symptom ontology. The probability determination module 720 may apply the techniques described above to search the medical symptom ontology based on a search query or tokenized terms of the search query to identify medical symptoms related to the search query. In some embodiments, the probability determination module 720 determines a patient-specific risk score for each medical condition identified from the medical condition ontology based at least in part on medical symptoms identified in the medical symptom ontology. Accordingly, in some embodiments, medical conditions in the medical condition ontology are mapped to symptoms in the medical symptom ontology, for example as illustrated in FIG. 6.

For example (not shown), a node for sensation perception may represent symptoms related to a patient's physical senses (e.g., touch, taste, smell, hearing, sight). In such a partial ontology, the primary node for sensation perception branches into a sublevel of secondary nodes, for example hyperesthesia, hypoesthesia, hyperalgesia, hypoalgesia, and pain. The secondary node for pain branches into nodes describing types of pain, for example abdominal pain and chest pain. The node for abdominal pain further branches into a sub-level of more specific nodes including epigastric abdominal pain, generalized abdominal pain, and periumbilic abdominal pain. The node for chest pain further branches into a sublevel of more specific nodes including pleuritic chest pain, precordial pain, and severe chest pain.

As another example (not shown), a node for sensation perception may represent neurological symptoms. In such a partial ontology, the primary node for neurological symptoms branches into a sublevel of secondary nodes, for example alteration of consciousness, dizziness, and fatigue. The secondary node for alteration of consciousness may further branch into a sub-level of specific types of alterations, for example coma, faint, stupor, and syncope. The secondary for dizziness may further breach into a sub-level of specific types of alterations, for example vertigo. The vertigo node may further branch into nodes for types of vertigo, for example objective vertigo and subjective vertigo. The secondary node for fatigue may further branch into a sub-level of specific types of fatigue, for example exhaustion, lethargy, tiredness, and weariness.

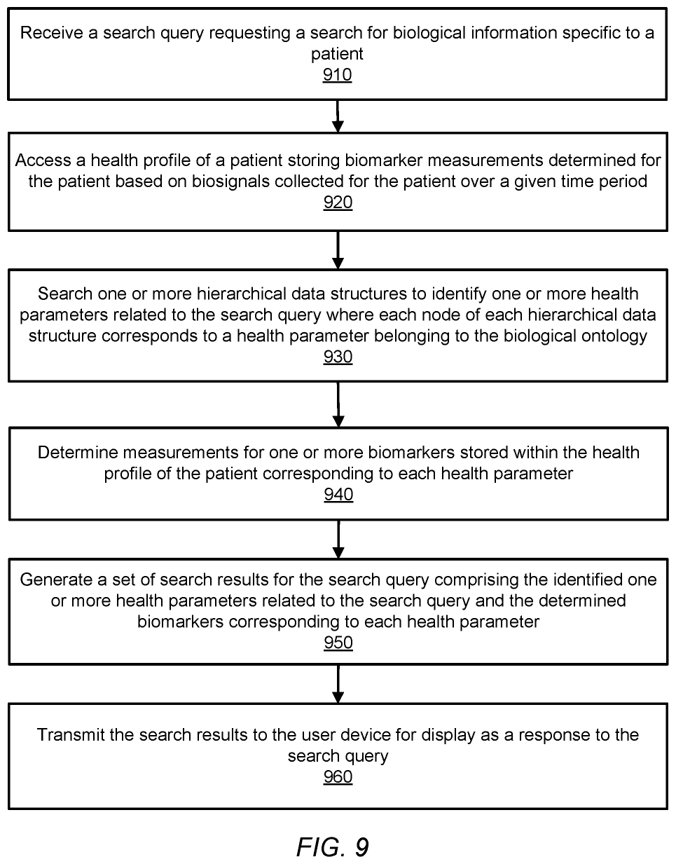

FIG. 9 illustrates an example process for performing a biological parameter search, according to one embodiment. In other embodiments, more, fewer or different steps may be included in the method. The precision health search module 260 receives 910 a search query input by a user through a health management application requesting a search for biological information specific to a patient. The search query may comprise biological information about which the user wishes to conduct a search specific to a patient. The precision health search module 260 may optionally parse the strength string using an NLP model to classify the search into a search type, separate the search string into tokens, or both. The precision health search module 260 accesses 920 aa health profile of a patient through the health management application. The health profile stores biomarkers determined for the patient based on biosignals collected for the patient over a given time period.

The precision health search module 260 searches 930 hierarchical data structures to identify one or more health parameters related to the search query. Each hierarchical data structure comprises a plurality of nodes organized into one or more levels of a biological ontology related to the search query. Each node corresponds to the health parameter belonging to the biological ontology. To search each hierarchical data structure, the precision health search module 260 identifies one or more primary nodes based on the search query and traverses the data structure to identify one or more secondary nodes linked to each primary node. One hierarchical data structure is a biomarker ontology of health parameters corresponding to measured biosignals, which the precision health search module 310 searches 930 to identify one or more health parameters related to the search query. Another hierarchical data structure is a nutrition ontology of health parameters corresponding to nutrition information, which the precision health search module 310 searches 930 to identify one or more health parameters related to the search query.

The precision health search module 260 determines 940 measurements for one or more biomarkers stored within the health profile of the patient (e.g., the digital twin module 250) corresponding to the one or more identified health parameters. The precision health search module 260 generates 950 a set of search results for the search query. The search results include the identified one or more health parameters related to the search query and the determined biomarkers corresponding to each health parameter. The precision health search module 260 transmits 960 the search results to the user device as a response to the search query for display via a graphical user interface.

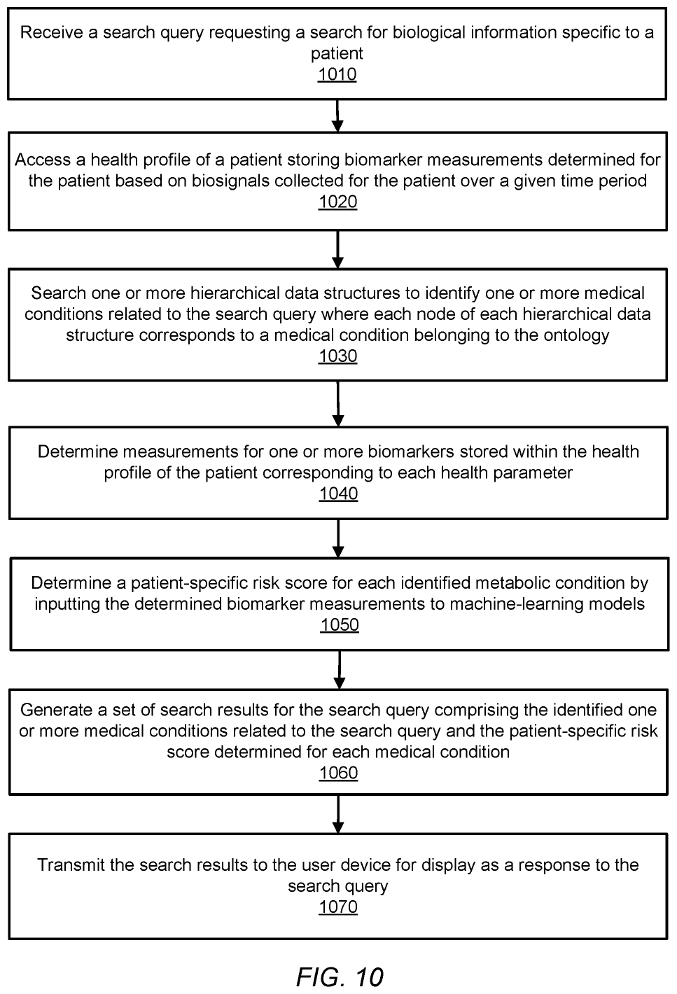

FIG. 10 illustrates an example process for performing a medical condition search, according to one embodiment. In other embodiments, more, fewer or different steps may be included in the method. The precision health search module 260 receives 1010 a search query input by a user through a health management application requesting a search for biological information specific to a patient. The search query may comprise biological information about which the user wishes to conduct a search specific to a patient. The precision health search module 260 may optionally parse the strength string using an NLP model to classify the search into a search type, separate the search string into tokens, or both. The precision health search module 260 accesses 1020 a health profile of a patient through the health management application. The health profile stores biomarkers determined for the patient based on biosignals collected for the patient over a given time period.

The precision health search module 260 searches 1030 hierarchical data structures to identify one or more medical conditions related to the search query. Each hierarchical data structure comprises a plurality of nodes organized into one or more levels of a biological ontology related to the search query. Each node corresponds to the health parameter belonging to the biological ontology. To search each hierarchical data structure, the precision health search module 260 identifies one or more primary nodes based on the search query and traverses the data structure to identify one or more secondary nodes linked to each primary node. Each hierarchical data structure is a medical condition ontology of health parameters corresponding to medical conditions, which the precision health search module 310 searches 1030 to identify one or more medical conditions related to the search query.

The precision health search module 260 determines 1040 measurements for one or more biomarkers stored within the health profile of the patient (e.g., the digital twin module 250) corresponding to the one or more identified health parameters. The precision health search module 260 determines 1050 a patient-specific risk score by inputting the determined biomarker measurements to a machine-learning model(s) trained to predict patient-specific risk scores for the one or more identified medical conditions. The precision health search module 260 generates 1060 a set of search results for the search query. The search results include the identified one or more medical conditions related to the search query and the patient-specific risk score determined for each medical condition. The precision health search module 260 transmits 1070 the search results to the user device as a response to the search query for display via a graphical user interface.

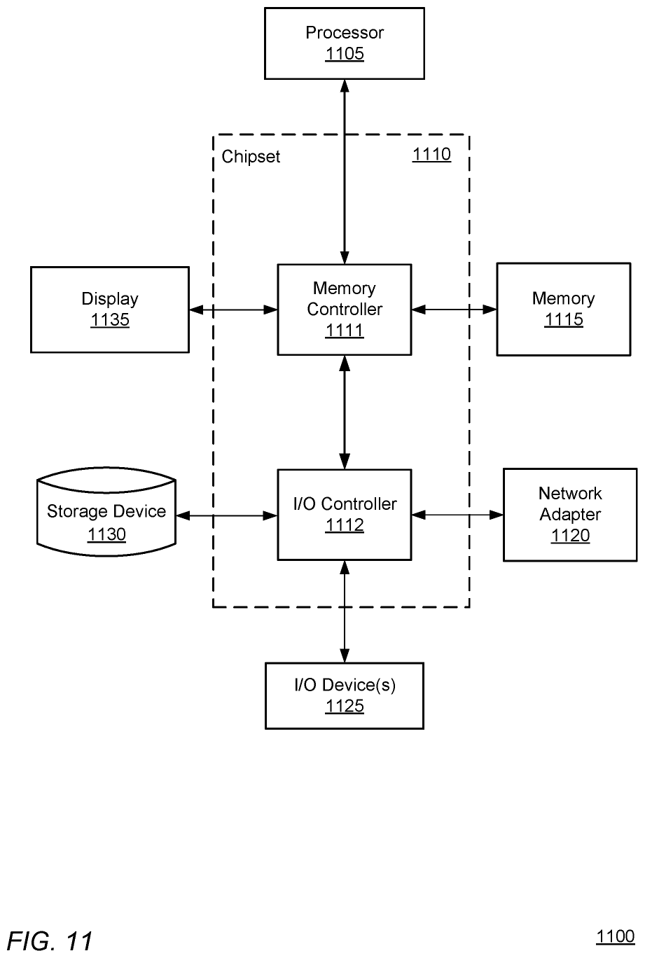

FIG. 11 is a high-level block diagram illustrating physical components of an example computer 1100 that may be used as part of a client device 110, application server 130, and/or database server 140 from FIG. 1, according to one embodiment. Illustrated is a chipset 1110 coupled to at least one processor 1105. Coupled to the chipset 1110 is volatile memory 1115, a network adapter 1120, an input/output (I/O) device(s) 1125, a storage device 1130 representing a non-volatile memory, and a display 1135. In one embodiment, the functionality of the chipset 1110 is provided by a memory controller 1111 and an I/O controller 1112. In another embodiment, the memory 1115 is coupled directly to the processor 1105 instead of the chipset 1110. In some embodiments, memory 1115 includes high-speed random-access memory (RAM), such as DRAM, SRAM, DDR RAM or other random access solid state memory devices.

The storage device 1130 is any non-transitory computer-readable storage medium, such as a hard drive, compact disk read-only memory (CD-ROM), DVD, or a solid-state memory device. The memory 1115 holds instructions and data used by the processor 1105. The I/O device 1125 may be a touch input surface (capacitive or otherwise), a mouse, track ball, or other type of pointing device, a keyboard, or another form of input device. The display 1135 displays images and other information for the computer 1100. The network adapter 1120 couples the computer 1100 to the network 160.

As is known in the art, a computer 1100 can have different and/or other components than those shown in FIG. 11. In addition, the computer 1100 can lack certain illustrated components.

In one embodiment, a computer 1100 acting as server 140 may lack a dedicated I/O device 1125, and/or display 1118. Moreover, the storage device 1130 can be local and/or remote from the computer 1100 (such as embodied within a storage area network (SAN)), and, in one embodiment, the storage device 1130 is not a CD-ROM device or a DVD device.

Generally, the exact physical components used in a client device 110 will vary in size, power requirements, and performance from those used in the application server 130 and the database server 140. For example, client devices 110, which will often be home computers, tablet computers, laptop computers, or smart phones, will include relatively small storage capacities and processing power, but will include input devices and displays. These components are suitable for user input of data and receipt, display, and interaction with notifications provided by the application server 130. In contrast, the application server 130 may include many physically separate, locally networked computers each having a significant amount of processing power for carrying out the asthma risk analyses introduced above. In one embodiment, the processing power of the application server 130 provided by a service such as Amazon Web Services™. Also in contrast, the database server 140 may include many, physically separate computers each having a significant amount of persistent storage capacity for storing the data associated with the application server.

As is known in the art, the computer 1100 is adapted to execute computer program modules for providing functionality described herein. A module can be implemented in hardware, firmware, and/or software. In one embodiment, program modules are stored on the storage device 1130, loaded into the memory 1115, and executed by the processor 1105.

### ADDITIONAL CONSIDERATIONS

It is to be understood that the figures and descriptions of the present disclosure have been simplified to illustrate elements that are relevant for a clear understanding of the present disclosure, while eliminating, for the purpose of clarity, many other elements found in a typical system. Those of ordinary skill in the art may recognize that other elements and/or steps are desirable and/or required in implementing the present disclosure. However, because such elements and steps are well known in the art, and because they do not facilitate a better understanding of the present disclosure, a discussion of such elements and steps is not provided herein. The disclosure herein is directed to all such variations and modifications to such elements and methods known to those skilled in the art.

Some portions of the above description describe the embodiments in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like.

Any of the steps, operations, or processes described herein may be performed or implemented with one or more hardware or software modules, alone or in combination with other devices. In one embodiment, a software module is implemented with a computer program product including a computer-readable non-transitory medium containing computer program code, which can be executed by a computer processor for performing any or all of the steps, operations, or processes described.

Embodiments of the invention may also relate to a product that is produced by a computing process described herein. Such a product may include information resulting from a computing process, where the information is stored on a non-transitory, tangible computer readable storage medium and may include any embodiment of a computer program product or other data combination described herein.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the invention. This description should be read to include one or at least one and the singular also includes the plural unless it is obvious that it is meant otherwise.

While particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

What is claimed is:

1. A method for performing a biological information search using a digital representation of a patient, the method comprising:
receiving, from a user of a health management application on a user device, a search query requesting a search for biological information specific to a patient;
accessing, through the health management application, a health profile of a patient storing biomarkers determined for the patient based on biosignals collected for the patient over a given time period;
searching, by the health management application, one or more hierarchical data structures to identify one or more health parameters related to the search query, each hierarchical data structure comprising a plurality of nodes organized into one or more levels of a biological ontology related to the search query, wherein each node corresponds to a health parameter belonging to the biological ontology;
determining, by the health management application, measurements for one or more biomarkers stored within the health profile of the patient corresponding to the one or more identified health parameters;
generating a set of search results for the search query comprising the identified one or more health parameters related to the search query and determined measurements corresponding to each health parameter; and
transmitting, for display via a graphical user interface, the search results to the user device as a response to the search query.

2. The method of claim 1, further comprising:
parsing, by a natural language processing (NLP) model, the search query into a string of tokens; or
classifying, by the NLP model, the search query into a search type.

3. The method of claim 1, further comprising:
mapping each node of each hierarchical data structure to one or more biomarkers stored within the health profile of the patient.

4. The method of claim 1, wherein the one or more levels of each hierarchical data structure are organized in order of increasing specificity such that a primary node in a higher level represents a category of health parameters describing secondary nodes in a lower level connected to the primary node.

5. The method of claim 1, wherein the one or more hierarchical data structures comprise a first biomarker ontology of health parameters corresponding to measurement biomarkers, the method further comprising:
searching the first biomarker ontology to identify one or more health parameters related to the search query; and
accessing measurements collected for each identified health parameter from the health profile of the patient, wherein the accessed measurements are collected by one or more of: a wearable sensor worn by the patient and lab test data collected for the patient.

6. The method of claim 1, wherein the one or more hierarchical data structures comprise a second biomarker ontology of health parameters corresponding to nutrition information, the method further comprising:
searching the second biomarker ontology to identify one or more health parameters related to the search query; and
determining measurements for each identified health parameter by inputting nutrition information collected for the patient to a machine-learning model trained to predict measurements for health parameters based on nutrition information.

7. The method of claim 1, wherein the one or more hierarchical data structures comprise a third biomarker ontology of medical conditions, the method further comprising:
searching the third biomarker ontology to identify one or more medical conditions related to the search query; and
determining, for each of the one or more identified medical conditions, a patient-specific risk score by inputting the determined biomarkers measurements to machine-learning models trained to predict patient-specific risk scores for the one or more identified medical conditions.

8. The method of claim 7, wherein the patient-specific risk score for each of the one or more identified medical conditions describes a probability that the patient is affected by the identified medical condition.

9. The method of claim 7, further comprising:
generating a patient-specific treatment recommendation outlining objectives for the patient to complete to improve their health profile based on the determined patient-specific risk scores.

10. The method of claim 7, wherein the graphical user interface displaying the search results further displays:
for each of the one or more identified health parameters,
graphical element displaying a trend in measurements for the health parameter recorded for the patient; and
a normal range of measurements corresponding to the health parameter.

11. A non-transitory computer-readable storage medium storing instructions that when executed cause a processor to:
receive, from a user of a health management application on a user device, a search query requesting a search for biological information specific to a patient;
access a health profile of a patient storing biomarkers determined for the patient based on biosignals collected for the patient over a given time period;
search one or more hierarchical data structures to identify one or more health parameters related to the search query, each hierarchical data structure comprising a plurality of nodes organized into one or more levels of a biological ontology related to the search query, wherein each node corresponds to a health parameter belonging to the biological ontology;
determine measurements for one or more biomarkers stored within the health profile of the patient corresponding to the one or more identified health parameters;
generate a set of search results for the search query comprising the identified one or more health parameters related to the search query and determined measurements corresponding to each health parameter; and
transmit, for display via a graphical user interface, the search results to the user device as a response to the search query.

12. The non-transitory computer-readable storage medium of claim 11, further comprising instructions that cause the processor to:
parse, by a natural language processing (NLP) model, the search query into a string of tokens; or
classify, by the NLP model, the search query into a search type.

13. The non-transitory computer-readable storage medium of claim 11, further comprising instructions that cause the processor to:
map each node of each hierarchical data structure to one or more biomarkers stored within the health profile of the patient.

14. The non-transitory computer-readable storage medium of claim 11, wherein the one or more levels of each hierarchical data structure are organized in order of increasing specificity such that a primary node in a higher level represents a category of health parameters describing secondary nodes in a lower level connected to the primary node.

15. The non-transitory computer-readable storage medium of claim 11, wherein the one or more hierarchical data structures comprise a first biomarker ontology of health parameters corresponding to measurement biomarkers, the non-transitory computer-readable storage medium further comprising instructions that cause the processor to:
search the first biomarker ontology to identify one or more health parameters related to the search query; and
access measurements collected for each identified health parameter from the health profile of the patient, wherein the accessed measurements are collected by one or more of: a wearable sensor worn by the patient and lab test data collected for the patient.

16. The non-transitory computer-readable storage medium of claim 11, wherein the one or more hierarchical data structures comprise a second biomarker ontology of health parameters corresponding to nutrition information, the non-transitory computer-readable storage medium further comprising instructions that cause the processor to:
search the second biomarker ontology to identify one or more health parameters related to the search query; and
determine measurements for each identified health parameter by inputting nutrition information collected for the patient to a machine-learning model trained to predict measurements for health parameters based on nutrition information.

17. The non-transitory computer-readable storage medium of claim 11, wherein the one or more hierarchical data structures comprise a third biomarker ontology of medical conditions, the non-transitory computer-readable storage medium further comprising instructions that cause the processor to:
search the third biomarker ontology to identify one or more medical conditions related to the search query; and
determine, for each of the one or more identified medical conditions, a patient-specific risk score by inputting the determined biomarkers measurements to machine-learning models trained to predict patient-specific risk scores for the one or more identified medical conditions.

18. The non-transitory computer-readable storage medium of claim 17, further comprising:
generating a patient-specific treatment recommendation outlining objectives for the patient to complete to improve their health profile based on the determined patient-specific risk scores.

19. The non-transitory computer-readable storage medium of claim 17, wherein the graphical user interface displaying the search results further displays:
for each of the one or more identified health parameters,
graphical element displaying a trend in measurements for the health parameter recorded for the patient; and
a normal range of measurements corresponding to the health parameter.

20. A system comprising:
a wearable sensor worn by a patient, the wearable sensor configured to collected sensor data during a current time period;
an application stored on a user device that presents search results to a user; and
a non-transitory computer-readable storage medium storing instructions that when executed cause a processor to:
receive, from the user of a health management application on the user device, a search query requesting a search for biological information specific to the patient;
access a health profile of a patient storing biomarkers determined for the patient based on biosignals collected for the patient over a given time period;
search one or more hierarchical data structures to identify one or more health parameters related to the search query, each hierarchical data structure comprising a plurality of nodes organized into one or more levels of a biological ontology related to the search query, wherein each node corresponds to a health parameter belonging to the biological ontology;
determine measurements for one or more biomarkers stored within the health profile of the patient corresponding to the one or more identified health parameters;
generate a set of search results for the search query comprising the identified one or more health parameters related to the search query and determined measurements corresponding to each health parameter; and
transmit, for display via a graphical user interface, the search results to the user device as a response to the search query.
